(19) United States
(12) Patent Application Publication
(10) Pub. No.: US 2007/0298079 A1
(43) Pub. Date: Dec. 27, 2007

(54) Food fortified with omega-3 fatty acids

(75) Inventors: Teodoro Rivera, Algonquin, IL (US); Peter S. Given, Ridgefield, CT (US); Nicholas C. Shields, Crystal Lake, IL (US); Amenah I. Ibrahim, Burbank, IL (US); Bryan Hitchcock, Vernon Hills, IL (US)

Correspondence Address: BANNER & WITCOFF, LTD.;and ATTORNEYS FOR CLIENT NO. 006943, 10 SOUTH WACKER DR., SUITE 3000, CHICAGO, IL, 60606, US

(73) Assignee: Tropicana Products, Inc., Bradenton, FL (US)

(21) Appl. No.: 11/474,928

(22) Filed: Jun. 26, 2006

(51) Int. Cl. A61K 47/00; A61K 31/202; A23D 7/00

(52) U.S. Cl. 424/439; 514/560; 426/601

(57) ABSTRACT

A food product is provided which is fortified with omega-3 fatty acids. The omega-3 fatty acids include docosahexaenoic acid (DHA) and eicosapentaenoic acid (EPA). The omega-3 fatty acids are added to the food product in a desired amount and blended sufficiently by mixing.

### FIELD OF THE INVENTION

The present invention relates generally to food products and, more specifically, to food products fortified with omega-3 fatty acids.

### BACKGROUND OF THE INVENTION

Two families of fatty acids, the omega-3 and the omega-6, form an important part of the human diet. Referred to as “essential fatty acids,” they constitute important components of cell membranes, regulate the body's use of cholesterol, and control the production of substances that affect nearly all other bodily processes. For example, eicosapentaenoic acid (EPA) and docosahexaenoic acid (DHA), long-chain forms of omega-3 fatty acids, support brain and cardiovascular health and functions, amongst other health benefits.

To achieve the best effect, the two families of fatty acids should be combined in the right proportions. Unlike omega-6 fatty acids, which are found in plant or vegetable oils and form a common part of a modem-day diet, both EPA and DHA are found almost exclusively in deep, cold water fish. The fact that omega-6 is more readily available than omega-3 has resulted in most people consuming a disproportionately low amount of omega-3 fatty acids. To increase or optimize health benefits from the essential fatty acids, it has been suggested that consumption of omega-3 fatty acids be increased, while that of omega-6 fatty acids be reduced.

From the above discussion, it is desirable to make omega-3 fatty acids, particularly long-chain forms, more readily available.

### SUMMARY OF THE INVENTION

The present invention relates to food products that promote health benefits, for example, a beverage fortified with omega-3 fatty acids. The beverage can be, for example, a not-from-concentrate (NFC) orange juice. The beverage can also include from-concentrate (FC) juices and other types of citrus or non-citrus juices, for example, 100% juices (e.g., apple and grape) and 1% to 90% juice cocktails (e.g., cranberry and grapefruit). Other beverages include, for example, dairy drinks, energy drinks, sports drinks, fortified/enhanced water drinks, soy drinks, fermented drinks (e.g., yogurt and kefir), carbonated drinks, hybrid mixtures of juice and dairy drinks, and the like, including both bottle and can products and fountain syrup applications. In addition, fortifying foods and beverages with omega-3 fatty acids is more convenient and fitting with modem lifestyles, as opposed to occasionally preparing and consuming fish.

In one aspect, the invention relates to a method for producing a food product. The method includes the steps of pre-processing to form an intermediate food product, adding a desired amount of omega-3 fatty acids to the intermediate food product, mixing the intermediate food product to disperse the omega-3 fatty acids in the intermediate food product. Optionally, the method can include the steps of pasteurizing the intermediate food product to form a food product and post-processing the food product. Post-processing may include preparing the product for packaging. The intermediate food product could be a solution or a semi-solid or solid mixture.

In another aspect, the invention relates to a food product including a product mixture and a desired amount of omega-3 fatty acids dispersed in the product mixture by mixing. In one embodiment, the product mixture is pasteurized. The product mixture could be a solution or a semi-solid or solid mixture.

In various embodiments of the foregoing aspects, the adding step includes adding a powder to the intermediate food product by, for example, using a powder mixer. The mixing step can include dispersing the omega-3 fatty acids within the intermediate food product to form a substantially homogeneous blend using, for example, a high shear mixer. In addition, the desired amount of omega-3 fatty acids is about 5-5000 milligrams per serving of the food product. In a particular embodiment, the desired amount of omega-3 fatty acids is at least about 16 milligrams per serving of the food product. In other embodiments, the desired amount of omega-3 fatty acids is about 5-320 milligrams, preferably about 15-100 milligrams, and more preferably about 10-50 milligrams per serving of the food product. The omega-3 fatty acids can include eicosapentaenoic acid (EPA) and/or docosahexaenoic acid (DHA) in any combination. In one embodiment, the omega-3 fatty acids include EPA and DHA in a ratio of about 60% EPA and about 40% DHA; however, it is possible to include only EPA or DHA in the food product.

These and other objects, along with advantages and features of the present invention herein disclosed, will become apparent through reference to the following description and the accompanying drawings. Furthermore, it is to be understood that the features of the various embodiments described herein are not mutually exclusive and can exist in various combinations and permutations.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention. In the following description, various embodiments of the present invention are described with reference to the following drawings, in which:

FIG. 1 shows a process for forming a food product in accordance with one embodiment of the invention.

### DETAILED DESCRIPTION OF THE INVENTION

The invention relates to a food product fortified with omega-3 fatty acids. In one embodiment, the food product comprises a liquid beverage. The liquid beverage, in one embodiment, comprises orange juice. The orange juice, for example, is a not-from-concentrate (NFC) orange juice. Food products comprising other types of beverages or juices, such as citrus, non-citrus, from concentrate, and not from concentrate, or others are also contemplated and within the scope of the invention. The food product could be in liquid form or non-liquid form. For example, the food product could be provided as a ready-to-drink beverage or in dry form for reconstituting with a liquid, such as water, for drinking. Additionally, the food product could be yogurt, oatmeal, cereal, cheese, pudding, rice cakes, snack bars, or other types of hand-held, non-refrigerated food products.

In one embodiment, the omega-3 fatty acids comprise the long-chain omega-3 fatty acids eicosapentaenoic acid (EPA) and docosahexaenoic acid (DHA). The long-chain omega-3 fatty acids are derived from, for example, marine or fish oils. Such oils can be extracted from various types of fish or marine animals, such as anchovies, capelin, cod, herring, mackerel, menhaden, salmon, sardines, shark and tuna, or from marine vegetation, such as micro-algae, or a combination thereof. Other sources of omega-3 fatty acids include liver and brain tissue and eggs.

The ratio of EPA to DHA in the orange juice or other food product may vary depending on the source of the omega-3 fatty acids (e.g., fish oils), the manner in which the omega-3 fatty acids are mixed, and the beverage/food product to be produced. The EPA:DHA ratio will vary to suit a particular application and can include, for example, 0:100, 100:0, 2:1, or 3:2. In a particular application the EPA:DHA ratio is 60:40; however, other ratios are contemplated and within the scope of the invention.

In one embodiment, the omega-3 fatty acids are added to the product (e.g., orange juice) in a powdered form and/or as a free oil in a liquid form. Additionally, the omega-3 fatty acid could be supplied in a gel form or as part of an emulsion with other types of carriers. Various types of powdered omega-3 fatty acids can be used. In one embodiment, the powdered form includes powdered omega-3 fatty acids prepared by spray-drying the fish oils into an encapsulation matrix comprising, for example, a protein and a carbohydrate. The matrix encapsulates the fish oils, forming microcapsules in the range of about 1-500 microns. In one embodiment, the encapsulate size range is about 5-100 microns. Additional ranges include about 0.5 to 20 microns. The particle size will be vary as necessary to suit a particular application and may be selected based on desired mouthfeel, visual appearance (hazy, cloudy, or opaque), oxidation stability, and suspension stability within the product. Encapsulated and multi-encapsulated fish oils include, for example, Meg-3® from Ocean Nutrition Canada. Encapsulated fish oils are advantageous, since the microcapsules can effectively protect the omega-3 fatty acids from stresses encountered due to oxidation of fatty acids during processing, such as, shear mixing, high temperature processing, and during subsequent storage.

A desired amount of omega-3 fatty acids is provided to the beverage/food product. The amount may vary depending on the application and nutritional content desired. In one embodiment, the orange juice comprises about 5-5000 mg of omega-3 fatty acids per 8 fluid ounces (0.24 liters)(serving size). The amount to be added will vary to suit a particular application and can be based, at least in part, on nutritional value, taste, shelf-life, efficacy levels approved, qualified health claims, and combinations thereof. Other amounts are also contemplated and within the scope of the invention. For example, it may be desired to provide at least 32 mg of omega-3 fatty acids (combined EPA and DHA) per serving of the beverage/food product to meet the United States Food and Drug Administration (FDA) excellent source nutrient content claim requirements, or 16 mg to meet the FDA good source nutrient content claim requirements. The omega-3 fatty acids are sufficiently mixed in the food product to provide a relatively uniform distribution; however, mixing is not limited to dissolving the omega-3 fatty acids in a liquid. For example, the omega-3 fatty acids may be mixed in powder form with a powdered drink mix (e.g., Gatorade® from Stokely-Van Camp, Inc.) to form a substantially evenly blended powdered product.

The beverage/food product may include other nutritional ingredients. For example, short-chain omega-3 fatty acids such as alpha-linolenic acid (ALA), which are derived from micro-algae or other sources, omega-6 fatty acids, vitamins, minerals, or a combinations thereof may also be added to the product. Ingredients such as sweeteners, flavorings, colorings, thickeners, emulsifiers, acidulants, electrolytes, proteins, carbohydrates, and preservatives can also be added to the product, as desired.

The finished food product with the omega-3 fatty acids may have a shelf-life of about 2-12 months and possibly up to 24 months under ambient conditions, depending on the level of processing the product undergoes, the type of packaging, and the materials used for packaging the product. In another embodiment (e.g., a more lightly processed product), the finished product with the omega-3 fatty acids may have a shelf-life of about 12 weeks under refrigerated conditions. Additional factors that may affect the shelf-life of the product include, for example, the nature of the base formula (e.g., a beverage sweetened with sugar has a longer shelf-life than a beverage sweetened with aspartame) and environmental conditions (e.g., exposure to high temperatures and sunlight is deleterious to ready to drink (RTD) beverages).

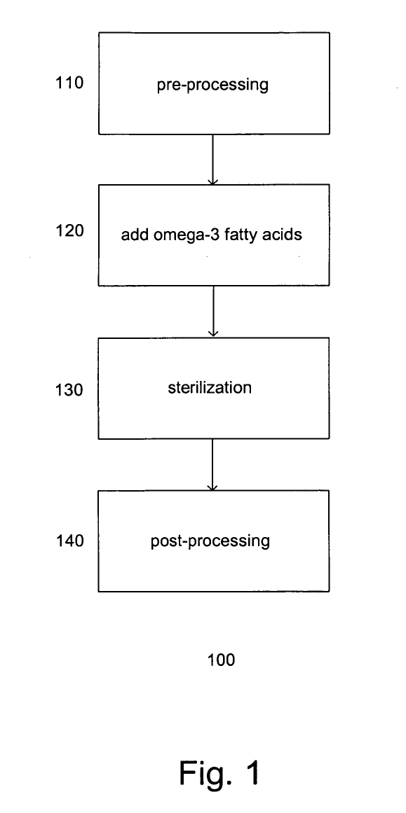

FIG. 1 shows a process 100 for forming a food product in accordance with one embodiment of the invention. At step 110, pre-processing is performed to provide an intermediate product. In one embodiment, the intermediate product is used to form orange juice, such as used to form NFC orange juice. Providing an intermediate product used to form other juices or beverages, such as those listed hereinabove, is also contemplated and within the scope of the invention.

In one embodiment, the pre-processing forms an intermediate food product just prior to pasteurization. Providing an intermediate food product at other stages of processing is also contemplated and within the scope of the invention. For example, the pre-processing can form an intermediate food product that requires additional processing prior to pasteurization. Various conventional techniques can be employed to form the intermediate food product. The pre-processing may vary depending on the application. In the case of NFC orange juice, this includes, for example, the addition of fortification or other additives and the lowering of the acidity levels of the juice, as disclosed in U.S. Pat. Nos. 6,565,898, 6,682,767, and 6,761,915, the entire disclosures of which are hereby incorporated herein by reference.

The intermediate food product can include various additional ingredients, such as vitamins, minerals, flavoring agents, sweeteners, coloring agents, other functional ingredients, stabilizers and pH adjusters, as desired. Other additives, such as those described hereinabove, are also contemplated and within the scope of the invention. Generally, the ingredients can be added prior to pasteurization, prior to mixing with the omega-3 fatty acid, and/or at the same time as the omega-3 fatty acid. The ingredients can also be added post-pasteurization.

In accordance with one embodiment of the invention, a desired amount of omega-3 fatty acids is added to the intermediate food product at step 120. The amount of omega-3 fatty acids added is, for example, about 5-5000 mg per 8 fluid ounces (0.24 liters) serving. Other amounts may also be useful, for example, depending on the serving size and nutritional content. In one embodiment, the omega-3 fatty acids in encapsulated or multi-encapsulated powder form are added to the intermediate food product. In another embodiment, the omega-3 fatty acids in encapsulated, multi-encapsulated slurry form, or as a free oil in a liquid form are added to the intermediate food product.

The omega-3 fatty acids are added to the intermediate food product after or during mixing of the intermediate food product. In one embodiment, high shear mixing is performed on the intermediate food product; however, other types of mixing are contemplated and within the scope of the invention, such as, for example, low energy/low shear mixing (e.g., stirring) and high energy/high shear mixing. The mixing can also be performed manually or as part of a batch process. Various types of high shear mixers can be employed. Typically, the high shear mixer includes rotatable blades enclosed in a housing. In one embodiment, the intermediate food product is made by use of two mixers. The first is a powder mixer that allows for incorporation of the ingredients in the intermediate food product. The second is an in-line high shear mixer. Mixing should be accomplished so as not to not destroy the encapsulation matrix, which might result in oxidation taking place. The mixer(s) can be selected for a specific application based, at least in part, on the type and amount of ingredients used, amount of product to be produced, and the flow rate. Generally, a commercially available mixer, such as those available from Invensys APV of Getzville, N.Y. or Silverson Machines, Inc. of East Longmeadow, Mass., may be used.

Many methods of incorporating the ingredients into the food product are available. In one embodiment, powder addition (or mixing) and high shear mixing is used. The powder mixer consists of a mixer-housing made up of an upper and lower chamber. The upper chamber has inlet connections for liquid and powder and the lower chamber has a special mixing-impeller mounted on a stainless steel shaft. The mixer operates by adding the dry ingredients into the hopper using a butterfly valve that controls the flow of powder from the hopper and prevents air from entering into the mixer. The dry powder gets sucked into the mixer where the impeller incorporates the ingredient into the intermediate food product stream. After passing the powder mixer, the intermediate food product then enters the high shear mixer. As the product is driven through the blades, the flow is repeatedly sheared through the action of the rotating blades. The high shear mixing disperses the omega-3 fatty acids sufficiently in the intermediate food product.

At step 130, the intermediate food product with omega-3 fatty acids is typically pasteurized. In one embodiment, pasteurization comprises pasteurizing the intermediate food product. The pasteurization process may include, for example, ultra high temperature (UHT) treatment and/or high temperature-short time (HTST) treatment. The UHT treatment includes subjecting the intermediate product solution to high temperatures, such as by direct steam injection or steam infusion, or by indirect heating in a heat exchanger. Generally, after the product is pasteurized, the product is optionally cooled as required by the particular product and/or the package filling application. For example, in one embodiment, the intermediate product solution is subjected to heating to about 185° F. (85° C.) to about 250° F. (121° C.) for a short period of time, for example, about 1 to 30 seconds, then cooled quickly to about 36° F. (2.2° C.) ±10° F. (5° C.) for refrigerated products, to ambient temperature for shelf stable or refrigerated products, and to about 185° F. (85° C.) ±10° F. (5° C.) for hot-fill applications for shelf-stable products. The pasteurization process is typically conducted in a closed system, so as not to expose the food product to atmosphere or other possible sources of contamination. Other pasteurization or sterilization techniques may also be useful, such as, for example, aseptic or retort processing. In addition, multiple pasteurization processes may be carried out in series or parallel, as necessitated by the food product or ingredients.

After processing is completed, post-processing is performed at step 140; however, post-processing may include any process steps carried out after the addition of the omega-3 fatty acids to the product. Post-processing includes, for example, cooling the product solution and filling it into containers for packaging and shipping. Post-processing may also include deaeration of the food product to <4.0 ppm oxygen, preferably <2.0 ppm, and more preferably <1.0 ppm; however, deaeration and other “post-processing” tasks may be carried out prior to processing, prior to pasteurization, prior to mixing with the omega-3 fatty acid, and/or at the same time as mixing the omega-3 fatty acid. In addition, an inert gas (e.g., nitrogen) headspace may be maintained during intermediary processing of the product and final packaging. Additionally or alternatively, an oxygen barrier and/or oxygen scavengers could be used in the final packaging. It is also contemplated and within the scope of the invention to include the process step of homogenizing the food product.

As described in accordance with one embodiment of the invention, the omega-3 fatty acids are incorporated into the food product in an encapsulated or powder form and subjected to high shear mixing. When compared with conventional methods, such as, for example, incorporating such components in oil form, the present invention gives rise to several significant advantages. For example, emulsifier need not be added to stabilize the fish oils, and no homogenization step is required during processing. In addition, reduction in the number of processing steps results in less off-flavors from oxidation of the omega-3 fatty acids, producing a more palatable product with a longer shelf-life.

The invention may be embodied in other specific forms without departing form the spirit or essential characteristics thereof. The foregoing embodiments, therefore, are to be considered in all respects illustrative rather than limiting the invention described herein. Scope of the invention is thus indicated by the appended claims, rather than by the foregoing description, and all changes that come within the meaning and range of equivalency of the claims are intended to be embraced therein.

What is claimed is:

1. A method for producing a food product comprising:
pre-processing to form an intermediate food product;
adding a desired amount of omega-3 fatty acids to the intermediate food product;
mixing the intermediate food product to disperse the omega-3 fatty acids in the intermediate food product and form the food product; and
post-processing the food product.

2. The method of claim 1, further comprising the step of pasteurizing the intermediate food product.

3. The method of claim 1, wherein the adding step comprises adding a powder to the intermediate food product.

4. The method of claim 1, wherein the mixing step comprises dispersing the omega-3 fatty acids within the intermediate food product to form a substantially homogeneous blend.

5. The method of claim 1, wherein the desired amount of omega-3 fatty acids comprises about 5-5000 milligrams per serving of the food product.

6. The method of claim 1, wherein the desired amount of omega-3 fatty acids comprises at least about 16 milligrams per serving of the food product.

7. The method of claim 1, wherein the omega-3 fatty acids comprise at least one of eicosapentaenoic acid (EPA) and docosahexaenoic acid (DHA).

8. The method of claim 7, wherein the omega-3 fatty acids comprise EPA and DHA in a ratio of about 60% EPA and about 40% DHA.

9. The method of claim 1, wherein the intermediate food product is selected from the group consisting of citrus juices, non-citrus juices, not-from-concentrate (NFC) juices, from concentrate (FC) juices, dairy drinks, energy drinks, sports drinks, fortified/enhanced water drinks, soy drinks, fermented drinks, carbonated drinks, hybrid mixtures of juice and dairy drinks, yogurt, oatmeal, cereal, pudding, cheese, rice cakes, snack bars, and combinations thereof.

10. A food product comprising:
a product mixture; and
a desired amount of omega-3 fatty acids dispersed in the product mixture by mixing.

11. The food product of claim 10, wherein the product mixture is pasteurized.

12. The food product of claim 10, wherein the mixing comprises adding a powder to the product mixture.

13. The food product of claim 10, wherein the mixing comprises dispersing the omega-3 fatty acids within the product mixture to form a substantially homogeneous blend.

14. The food product of claim 10, wherein the product solution is selected from the group consisting of citrus juices, non-citrus juices, not-from-concentrate (NFC) juices, from concentrate (FC) juices, dairy drinks, energy drinks, sports drinks, fortified/enhanced water drinks, soy drinks, fermented drinks, carbonated drinks, hybrid mixtures of juice and dairy drinks, yogurt, oatmeal, cereal, pudding, cheese, rice cakes, snack bars, and combinations thereof.

15. The food product of claim 10, wherein the desired amount of omega-3 fatty acids comprises about 5-5000 milligrams per serving of the food product.

16. The food product of claim 10, wherein the desired amount of omega-3 fatty acids comprises at least about 16 milligrams per serving of the food product.

17. The food product of claim 10, wherein the omega-3 fatty acids comprise at least one of eicosapentaenoic acid (EPA) and docosahexaenoic acid (DHA).

18. The food product of claim 17, wherein the omega-3 fatty acids comprise EPA and DHA in a ratio of about 60% EPA and about 40% DHA.
